(19) United States
(12) Patent Application Publication
(10) Pub. No.: US 2013/0218284 A1
(43) Pub. Date: Aug. 22, 2013

(54) TIBIAL BASEPLATE ASSEMBLY FOR KNEE JOINT PROSTHESIS

(71) Applicant: Mobius Medical LLC, (US)

(72) Inventors: Thomas Eickmann, Longmont, CO (US); Daniel E. E. Hayes, JR., Placerville, CA (US); Sterling R. Grover, Placerville, CA (US)

(73) Assignee: MOBIUS MEDICAL LLC, New Canaan, CT (US)

(21) Appl. No.: 13/770,926

(22) Filed: Feb. 19, 2013

Related U.S. Application Data
(60) Provisional application No. 61/599,816, filed on Feb. 16, 2012.
(60) Provisional application No. 61/730,836, filed on Nov. 28, 2012.

(51) Int. Cl. A61F 2/38

(52) U.S. Cl. CPC A61F 2/389
USPC 623/20.34; 623/20.32

(57) ABSTRACT

Apparatus for reconstructing a joint, the apparatus comprising:
an implant body having a bone contacting surface; and
a plurality of fixation elements secured to the implant body and extending into the host bone at a plurality of angles, wherein all angles are not equal to one another, so as to create immediate stability between the implant body and the host bone.

### REFERENCE TO PENDING PRIOR PATENT APPLICATIONS

This patent application claims benefit of:

(i) pending prior U.S. Provisional Patent Application Ser. No. 61/599,816, filed Feb. 16, 2012 by Thomas Eickmann et al. for TIBIAL BASE CONSTRUCT FOR KNEE JOINT PROSTHESIS (Attorney's Docket No. MOBIUS-1 PROV); and

(ii) pending prior U.S. Provisional Patent Application Ser. No. 61/730,836, filed Nov. 28, 2012 by Thomas Eickmann et al. for CEMENTLESS BASEPLATE (Attorney's Docket No. MOBIUS-5 PROV).

The two (2) above-identified patent applications are hereby incorporated herein by reference.

### FIELD OF THE INVENTION

This invention relates to orthopaedic prostheses in general, and more particularly to tibial baseplates for knee joint prostheses.

### BACKGROUND OF THE INVENTION

Joint replacement surgery seeks to replace some or all of a natural joint with prosthetic components so as to provide long-lasting function and pain-free mobility.

For example, in the case of a prosthetic total hip joint, the head of the femur is replaced with a prosthetic femoral stem component, and the socket of the acetabulum is replaced by a prosthetic acetabular cup component, whereby to provide a prosthetic total hip joint.

In the case of a prosthetic total knee joint, the top of the tibia is replaced by a prosthetic tibial component, and the bottom of the femur is replaced by a prosthetic femoral component, whereby to provide a prosthetic total knee joint.

The present invention is directed to orthopedic prostheses for restoring the knee joint and, in particular, to improved prosthetic tibial components.

Looking now at FIG. 1, there is shown a prior art prosthetic total knee joint 5 which generally comprises a prosthetic tibial component 10 secured to the top end of a resected tibia 15, and a prosthetic femoral component 20 which is secured to the bottom end of a resected femur 25.

A typical prior art prosthetic tibial component 10 is shown in greater detail in FIGS. 2 and 3. Prior art prosthetic tibial component 10 generally comprises a metal base construct 30 and a polyethylene bearing construct 35.

More particularly, metal base construct 30 generally comprises a baseplate 40 having a top surface 42 and a bottom surface 43, a stem 45 and a plurality of posts 50 descending from bottom surface 43 of baseplate 40 and into resected tibia 15, a plurality of screws 55 passing through baseplate 40 and into resected tibia 15, a pair of rails 60 running along top surface 42 of baseplate 40 and defining a groove 65 therebetween, and a pair of end walls 70 connected to top surface 42 of baseplate 40. Preferably bottom surface 43 of baseplate 40 (and, optionally, stem 45 and/or posts 50) comprises a porous material so as to allow bone ingrowth into baseplate 40 (and/or stem 45 and/or posts 50), whereby to facilitate osseo-integration of the baseplate (and/or stem 45 and/or posts 50) with resected tibia 15 over time.

Polyethylene bearing construct 35 comprises a flat bottom surface 75 having a recess 80 in which is disposed a tongue 85. Tongue 85 is sized to slidingly fit in groove 65 of metal base construct 30 (FIG. 3), whereby polyethylene bearing construct 35 may be slidingly secured to metal base construct 30. Note that end walls 70 act as stops for polyethylene bearing construct 35 when tongue 85 of polyethylene bearing construct 35 is advanced into groove 65 of metal base construct 30. It should be appreciated that the aforementioned “tongue-in-groove” locking mechanism between polyethylene bearing construct 35 and metal base construct 30 (i.e., tongue 85 of polyethylene bearing construct 35 and groove 65 of metal base construct 30) is only one of the many different locking mechanisms used in the art to secure a polyethylene bearing construct to a metal base construct, and is shown here for purposes of example but not limitation.

In use, the top end of tibia 15 is resected, and metal base construct 30 is secured to tibia 15, i.e., by advancing stem 45 and posts 50 into resected tibia 15 until bottom surface 43 of baseplate 40 is seated against resected tibia 15. Note that the parallel dispositions of stem 45 and posts 50 facilitates advancement of stem 45 and posts 50 into the resected tibia. Next, screws 55 are advanced through baseplate 40 and into resected tibia 15, whereby to secure metal base construct 30 to resected tibia 15. Then polyethylene bearing construct 35 is locked onto metal base construct 30, e.g., by sliding tongue 85 of polyethylene bearing construct 35 into groove 65 of metal base construct 30 until polyethylene bearing construct 35 engages end walls 70 of baseplate 40.

Ideally, baseplate 40, stem 45 and posts 50 osseo-integrate with resected tibia 15 over time, thereby providing stable attachment of prosthetic tibial component 10 to resected tibia 15.

Unfortunately, in many patients, micromotion of baseplate 40, stem 45 and posts 50 relative to resected tibia 15 inhibits osseo-integration. In addition, micromotion can lead to the development of a fibrous membrane at the interface of the prosthesis and the resected tibia. This fibrous membrane further inhibits bony ingrowth into the prosthesis, and the result is a loose and painful joint. In many patients, the consequences of the aforementioned micromotion between the prosthesis and the bone (i.e., lack of proper osseo-integration, development of a fibrous membrane between the prosthesis and the resected tibia, and the resulting pain for the patient) ultimately requires revision surgery.

As a result, in many cases, clinicians apply bone cement (typically polymethylmethacrylate, also known as PMMA) between bottom surface 43 of baseplate 40 and the top of resected tibia 15, and/or around stem 45 and/or posts 50. However, since this bone cement is interposed between the prosthesis and the tibia, it inhibits the aforementioned osseo-integration process, thereby reducing the possibility of long-term biological fixation via bony ingrowth. Furthermore, over time, bone cement can deteriorate, thereby causing loosening of the prosthesis, significant pain for the patient and, in many cases, ultimately requiring revision surgery.

Thus there is a need for a new and improved tibial baseplate assembly for a knee joint prosthesis which can provide for immediate cementless fixation of the tibial baseplate to the resected tibia while effectively eliminating micromotion between the prosthesis and the tibia, thereby providing an early post-operative environment where bony ingrowth can occur, providing long-term, biological fixation between bone and prosthesis which has the potential to provide a lifetime result for the patient.

### SUMMARY OF THE INVENTION

The present invention comprises the provision and use of a new and improved tibial baseplate assembly for a knee joint prosthesis which provides for immediate cementless fixation of the tibial baseplate to the resected tibia while effectively eliminating micromotion between the prosthesis and the tibia, thereby providing the environment for long-term biological fixation via bony ingrowth into the prosthesis surface.

The present invention also comprises the provision and use of a new and improved prosthetic tibial component which comprises a base construct and a bearing construct, wherein the base construct comprises a novel tibial baseplate assembly which provides for immediate cementless fixation of the tibial baseplate to the resected tibia while effectively eliminating micromotion between the prosthesis and the tibia, thereby providing the environment for long-term biological fixation via bony ingrowth into the prosthesis surface.

The present invention also comprises a novel method for reconstructing a knee joint, wherein the novel method comprises the provision and use of an improved prosthetic tibial component which comprises a base construct and a bearing construct, and further wherein the base construct comprises a novel tibial baseplate assembly which provides for immediate cementless fixation of the tibial baseplate to the resected tibia while effectively eliminating micromotion between the prosthesis and the tibia, thereby providing the environment for long-term biological fixation via bony ingrowth into the prosthesis surface.

In one preferred form of the invention, there is provided apparatus for reconstructing a joint, the apparatus comprising:

an implant body having a bone contacting surface; and

a plurality of fixation elements secured to the implant body and extending into the host bone at a plurality of angles, wherein all angles are not equal to one another, so as to create immediate stability between the implant body and the host bone.

In another preferred form of the invention, there is provided a method for reconstructing a joint, the method comprising:

providing apparatus comprising:
an implant body having a bone contacting surface; and
a plurality of fixation elements to be secured to the implant body and extend into the host bone at a plurality of angles, wherein all angles are not equal to one another, so as to create immediate stability between the implant body and the host bone;

positioning the implant body against a host bone; and

creating immediate stability between the implant body and the host bone using the plurality of fixation elements.

In another preferred form of the invention, there is provided apparatus for reconstructing the knee joint, the apparatus comprising:

a tibial baseplate; and

at least two fixation elements for securing the tibial baseplate to a bone, wherein the at least two fixation elements are secured to the tibial baseplate and extend into the tibia at angles which are not parallel to one another.

In another preferred form of the invention, there is provided a method for reconstructing the knee joint, the method comprising:

providing apparatus comprising:
a tibial baseplate; and
at least two fixation elements for securing the tibial baseplate to a bone, wherein the at least two fixation elements are secured to the tibial baseplate and extend into the tibia at angles which are not parallel to one another;

positioning the tibial baseplate against a resected tibia; and

securing the tibial baseplate to the resected tibia using the at least two fixation elements, wherein the at least two fixation elements are secured to the tibial baseplate and extend into the tibia at angles which are not parallel to one another.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic side view showing a prior art prosthetic total knee joint;

FIG. 2 is a schematic partially-exploded perspective view showing a prior art prosthetic tibial component;

FIG. 3 is a schematic front view showing a prior art prosthetic tibial component secured to a resected tibia;

FIG. 4 is a schematic front view showing a new and improved tibial baseplate assembly for a knee joint prosthesis;

FIG. 5 is a view like that of FIG. 4, except that the baseplate of the tibial baseplate assembly is shown semi-transparent;

FIG. 6 is a schematic lateral view showing the new and improved tibial baseplate assembly of FIG. 4;

FIG. 7 is a schematic posterior view showing the new and improved tibial baseplate assembly of FIG. 4;

FIG. 8 is a schematic medial view showing the new and improved tibial baseplate assembly of FIG. 4;

FIG. 9 is a schematic bottom view showing the new and improved tibial baseplate assembly of FIG. 4, with the baseplate of the tibial baseplate assembly being shown semi-transparent;

FIG. 10 is a schematic top view showing the new and improved tibial baseplate assembly of FIG. 4, with the baseplate of the tibial baseplate assembly being shown semi-transparent;

FIG. 11 is a partial schematic front view showing a peg secured to the baseplate of the new and improved tibial baseplate assembly of FIG. 4;

FIG. 11A is a schematic front view showing a template for use in forming holes in the resected tibia for receiving the pegs of the new and improved tibial baseplate assembly of FIG. 4;

FIG. 11B is a schematic front view showing a peg secured to the baseplate of the new and improved tibial baseplate assembly of FIG. 4 using a hole formed by the template shown in FIG. 11A;

FIG. 12 is a schematic view showing an alternative form of pegs secured to the baseplate of the new and improved tibial baseplate assembly of FIG. 4;

FIG. 13 is a schematic bottom view showing the components of FIG. 12;

FIG. 14 is a view like that of FIG. 12, except showing one of the pegs about to be secured to the baseplate;

FIG. 15 is a schematic perspective view showing pegs and a bone screw securing the baseplate of the new and improved tibial baseplate assembly to the resected tibia; and

FIG. 16 is a schematic lateral view showing pegs and a bone screw securing the baseplate of the new and improved tibial baseplate assembly to the resected tibia.

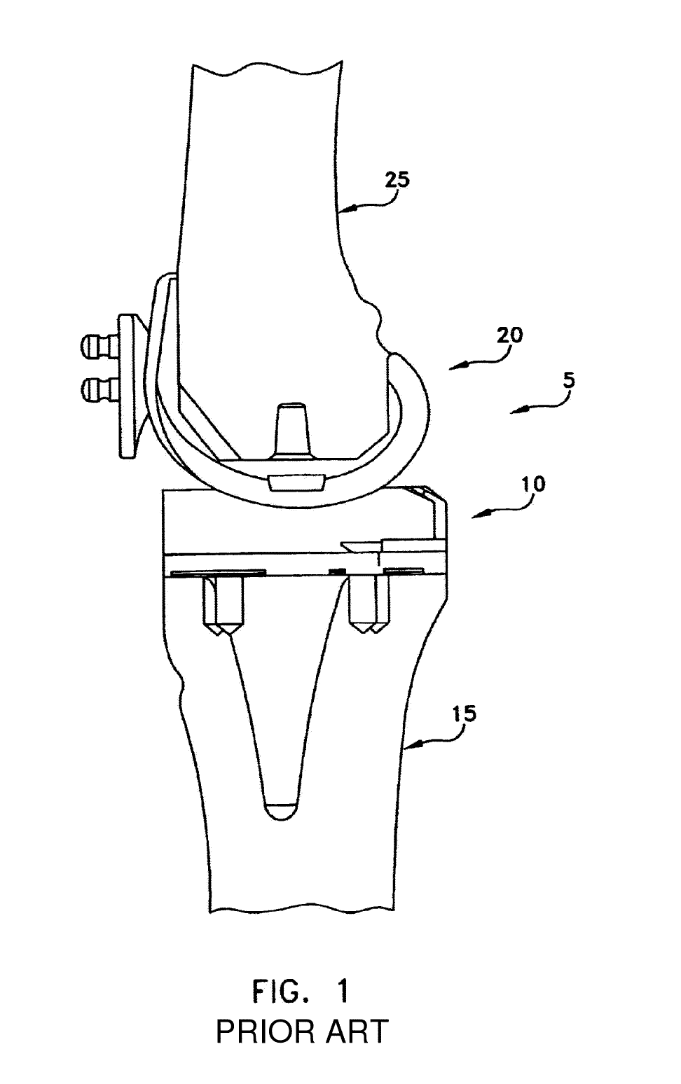

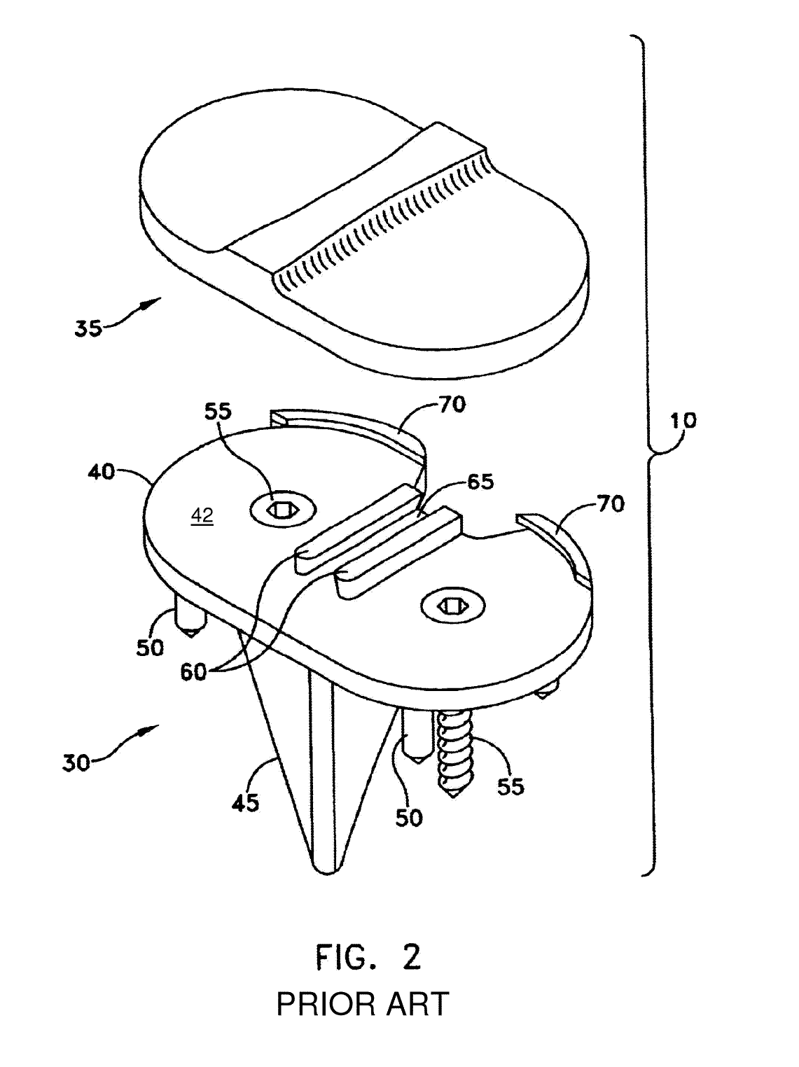

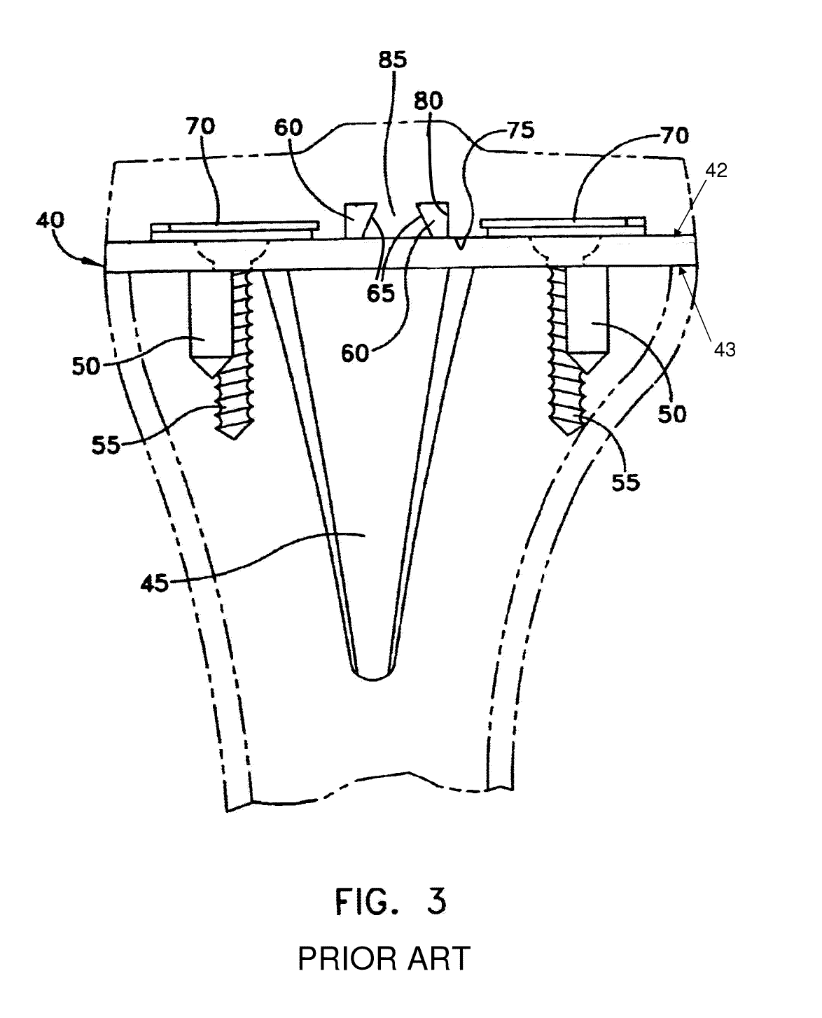

### DETAILED DESCRIPTION OF THE PREFERRED EMBODIMENTS

The present invention comprises the provision and use of a new and improved tibial baseplate assembly for a knee joint prosthesis which provides for immediate cementless fixation of the tibial baseplate to the resected tibia while effectively eliminating micromotion between the prosthesis and the tibia.

The present invention also comprises the provision and use of a new and improved prosthetic tibial component which comprises a base construct and a bearing construct, and further wherein the base construct comprises a novel tibial baseplate assembly which provides for immediate cementless fixation of the tibial baseplate to the resected tibia while effectively eliminating micromotion between the prosthesis and the tibia.

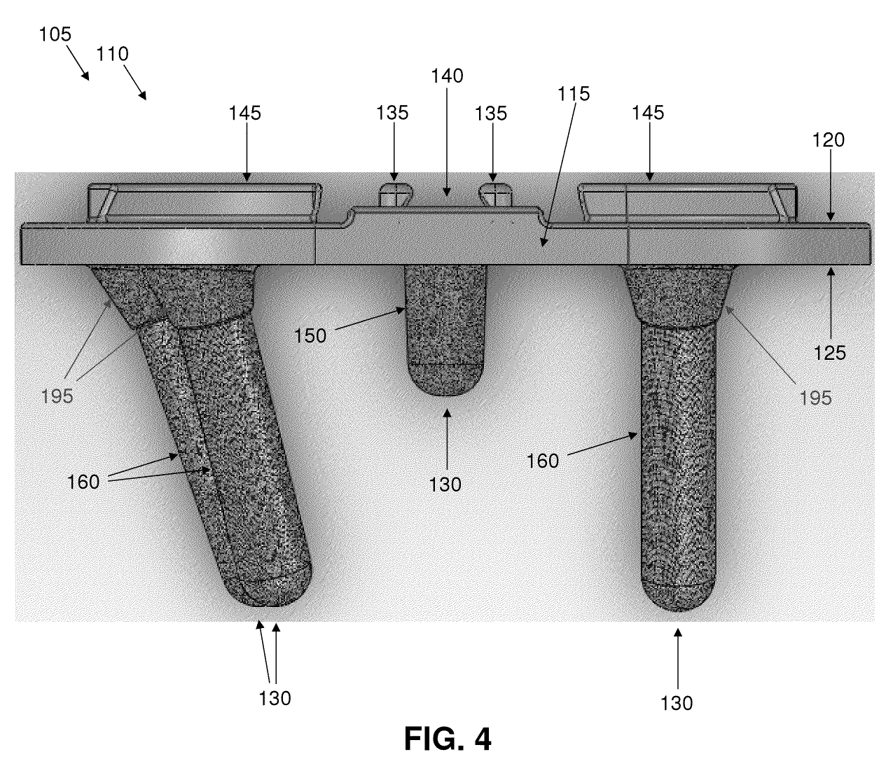

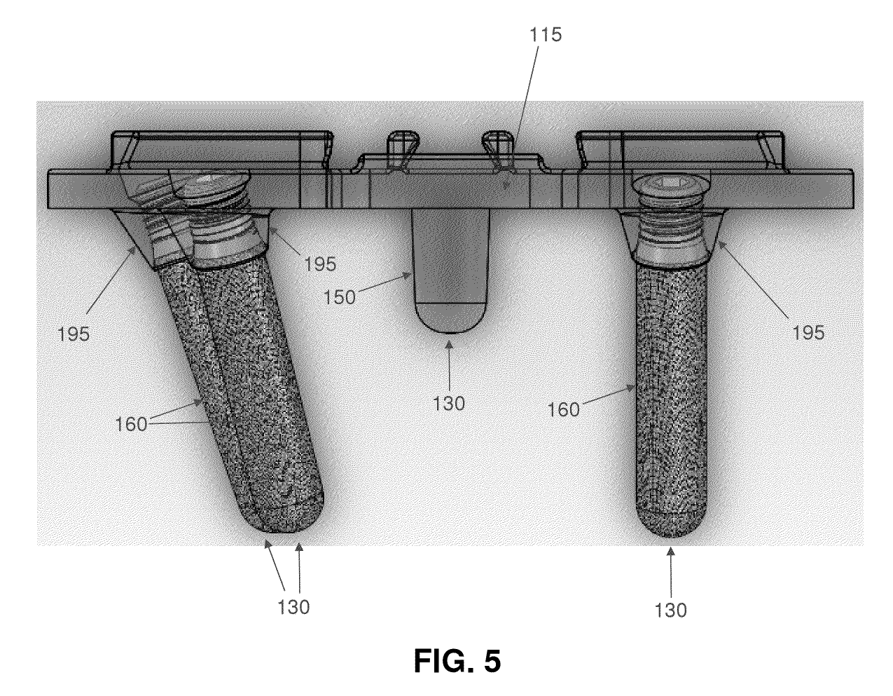

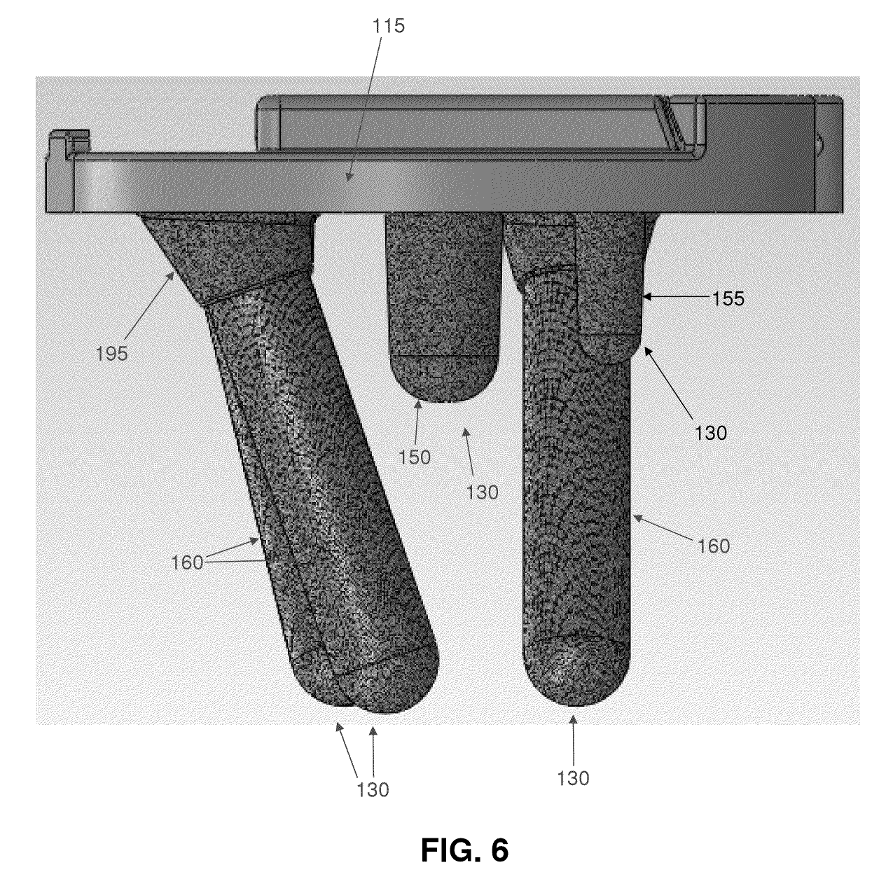

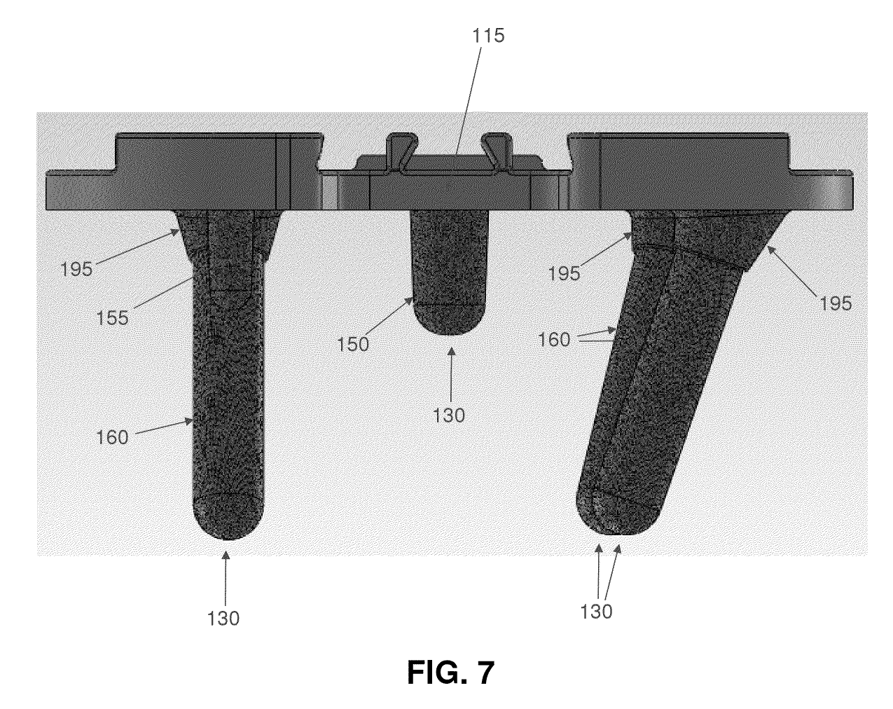

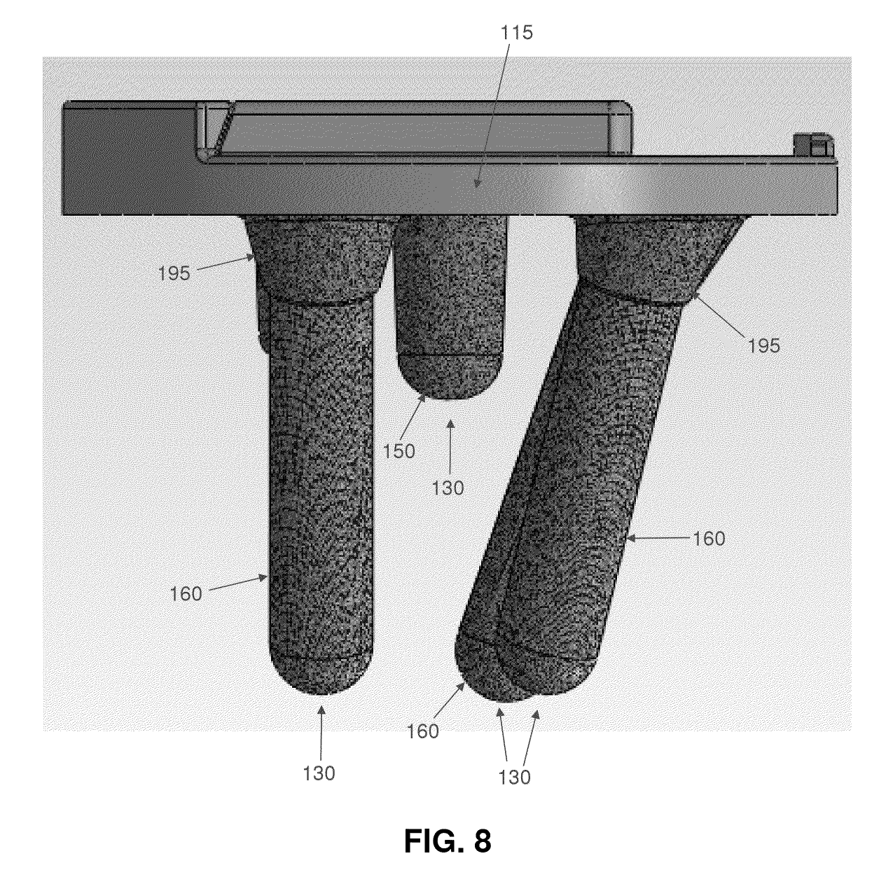

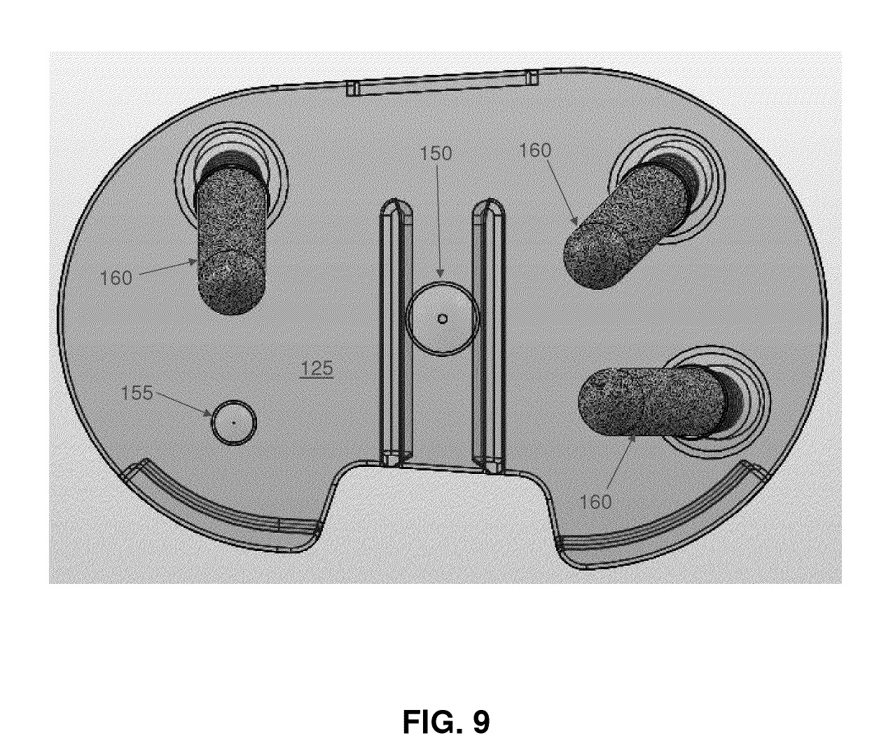

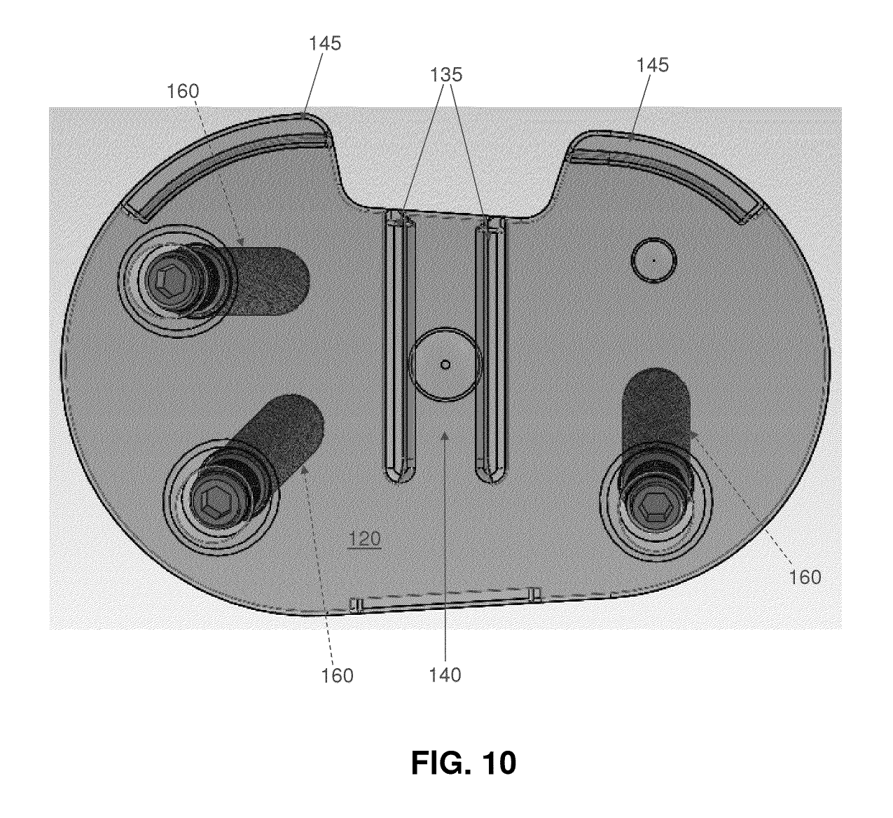

The present invention also comprises a novel method for reconstructing a knee joint, wherein the novel method comprises the provision and use of an improved prosthetic tibial component which comprises a base construct and a bearing construct, and further wherein the base construct comprises a novel tibial baseplate assembly which provides for immediate cementless fixation of the tibial baseplate to the resected tibia while effectively eliminating micromotion between the prosthesis and the tibia. Looking now at FIGS. 4-11, there is shown a novel base construct 105 for use in conjunction with a bearing construct (e.g., the polyethylene bearing construct 35 discussed above) so as to form a prosthetic tibial component for a prosthetic total knee joint.

Novel base construct 105 comprises a novel tibial baseplate assembly 110 for mounting to the resected tibia and receiving a bearing construct (e.g., the polyethylene bearing construct 35 discussed above). Novel tibial baseplate assembly 110 generally comprises a baseplate 115 having a top surface 120 and a bottom surface 125, and at least two fixation elements 130 extending downwardly from bottom surface 125 of baseplate 115 for receipt in a resected tibia (not shown) so as to cementlessly secure baseplate 115 (and hence novel base construct 105) to the resected tibia while effectively eliminating micromotion between the prosthesis and the tibia. In accordance with the present invention, and as will hereinafter be discussed in further detail, at least two of the fixation elements 130 extend downwardly from bottom surface 125 of baseplate 115 in a non-parallel manner. Such non-parallel disposition of at least two of the fixation elements 130 ensures that baseplate 115 is cementlessly secured to the tibia while effectively eliminating micromotion between the prosthesis and the tibia. Preferably bottom surface 125 of baseplate 115 (and, optionally, one or more of the at least two fixation elements 130) comprise a porous material so as to allow bone ingrowth into baseplate 115 (and/or one or more of the at least two fixation elements 130), whereby to facilitate osseo-integration of the baseplate 115 (and/or one or more of the at least two fixation elements 130) with the resected tibia over time.

A pair of rails 135 run along top surface 120 of baseplate 115 and define a groove 140 therebetween. A pair of end walls 145 are connected to top surface 120 of baseplate 115. Rails 135 and end walls 145 are intended to facilitate securing a bearing construct (e.g., such as the aforementioned polyethylene bearing construct 35) to baseplate 115 via a “tongue-in-groove” locking mechanism (e.g., via disposition of tongue 85 of polyethylene bearing construct 35 into groove 140 of baseplate 115), although such a “tongue-in-groove” locking mechanism is only one of the many different locking mechanisms which may be used to secure a bearing construct (such as the polyethylene bearing construct 35) to baseplate 115, and hence it should be appreciated that rails 135 and end walls 145 are shown here for purposes of example but not limitation.

As noted above, novel tibial baseplate assembly 110 comprises at least two fixation elements 130 extending downwardly from bottom surface 125 of baseplate 115 for receipt in a resected tibia (not shown) so as to cementlessly secure baseplate 115 (and hence novel base construct 105) to the resected tibia while effectively eliminating micromotion between the prosthesis and the tibia. In accordance with the present invention, at least two of the fixation elements 130 extend downwardly from bottom surface 125 of baseplate 115 in a non-parallel manner. Such non-parallel disposition of at least two of the fixation elements 130 ensures that baseplate 115 is cementlessly secured to the tibia while effectively eliminating micromotion between the prosthesis and the tibia.

In one preferred form of the invention, fixation elements 130 comprise a stem 150, a post 155 and a plurality of pegs 160.

Stem 150 is preferably formed integral with baseplate 115 and comprises a substantially straight body extending downwardly from bottom surface 125 of baseplate 115. Stem 150 extends at a right angle to bottom surface 125 of baseplate 115. Stem 150 may have a circular cross-section or other cross-section, straight or tapered sides or edges, and a pointed, rounded or flat distal end. Stem 150 is preferably located intermediate bottom surface 125 of baseplate 115 so as to extend into the intramedullary canal of the tibia when baseplate 115 is set on the resected tibia. In one preferred form of the invention, substantially the entire outer surface of stem 150 comprises a porous coating so as to facilitate osseo-integration of the surrounding bone into the stem.

Post 155 is preferably formed integral with baseplate 115 and comprises a substantially straight body extending downwardly from bottom surface 125 of baseplate 115. Post 155 extends at a right angle to bottom surface 125 of baseplate 115. Post 155 may have a circular cross-section or other cross-section, straight or tapered sides or edges, and a pointed, rounded or flat distal end. Post 155 is preferably located intermediate bottom surface 125 of baseplate 115 so as to extend into the tibia when baseplate 115 is set on the resected tibia. In one preferred form of the invention, substantially the entire outer surface of post 155 comprises a porous coating so as to facilitate osseo-integration of the surrounding bone into the stem.

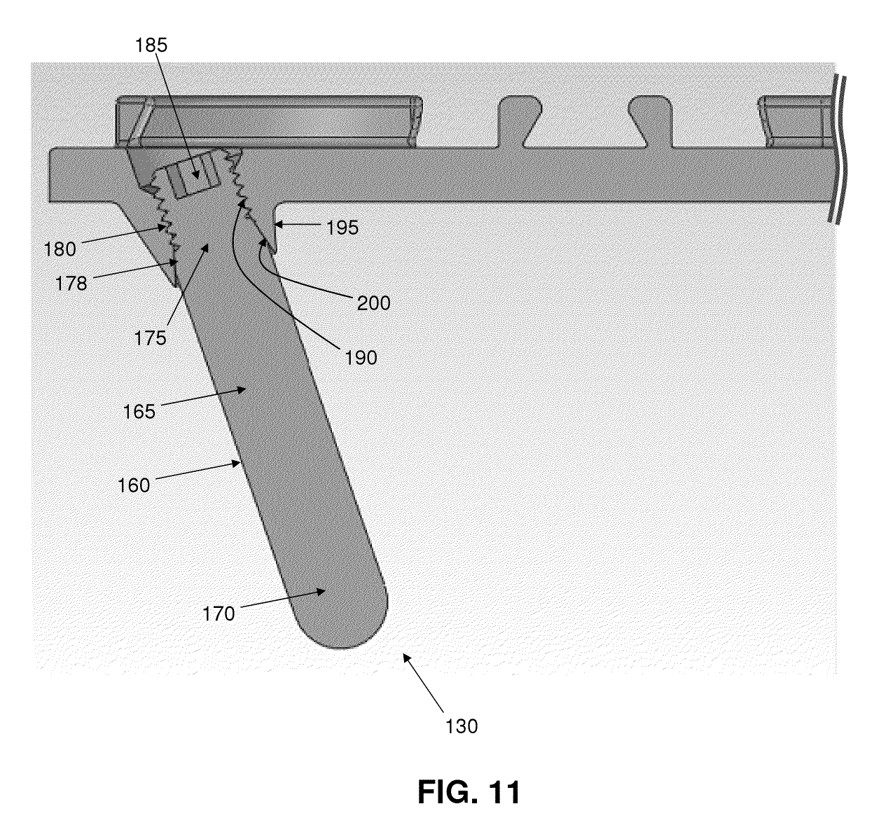

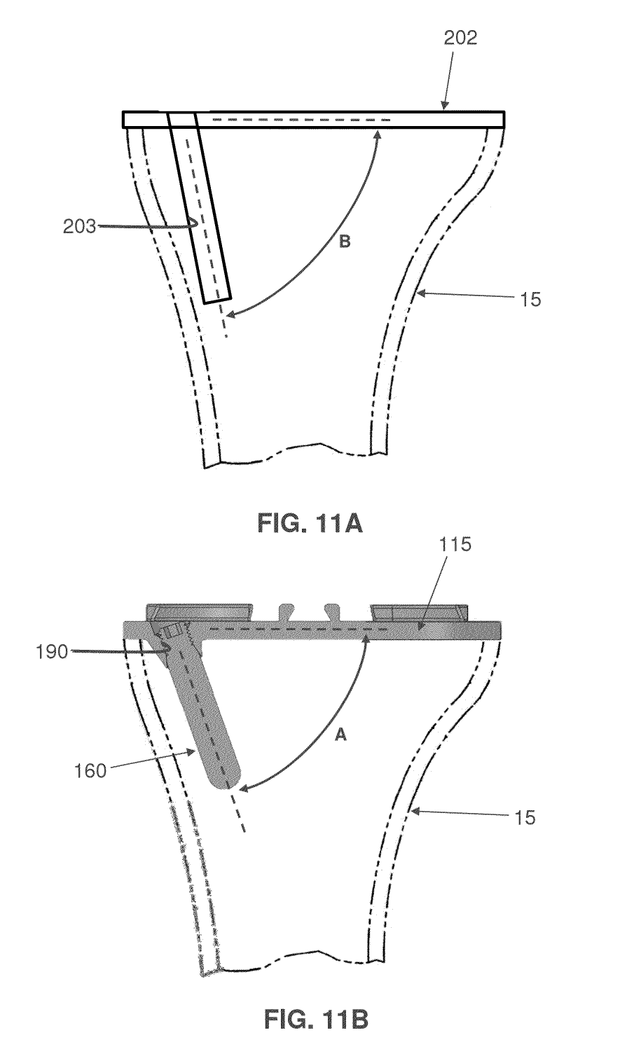

Each peg 160 comprises an elongated body 165 having a distal end 170 and a proximal end 175 (FIG. 11). Elongated body 165 preferably has a circular cross-section. Distal end 170 of elongated body 165 is preferably rounded so as to facilitate advancement into a hole formed in the tibia (see below), and proximal end 175 of elongated body 165 includes an inward taper 178 and an outer screw thread 180 proximal to inward taper 178. A non-circular recess 185, for receiving a driver (not shown), is formed in proximal end 175 of elongated body 165. Pegs 160 are intended to be releasably secured to baseplate 115 via the aforementioned screw thread 180 and, to this end, baseplate 115 comprises a plurality of threaded bores 190 extending therethrough. In accordance with one preferred form of the present invention, each threaded bore 190 extends at a non-perpendicular angle to the plane of baseplate 115, such that when pegs 160 are screwed into threaded bores 190, pegs 160 will extend at non-perpendicular angles to the plane of baseplate 115. Furthermore, in one preferred form of the invention, threaded bores 190 extend non-parallel to one another, such that pegs 160 also extend non-parallel to one another.

In order to provide sufficient stability to pegs 160, baseplate 115 preferably comprises collars 195 about each of the threaded bores 190, with collars 195 being formed integral with baseplate 115 and extending distally from baseplate 115, coaxial with threaded bores 190. Collars 195 terminate in a tapered shoulder 200 for mating with inward taper 178 of pegs 160.

In one preferred form of the invention, substantially the entire outer surface of pegs 160 comprise a porous coating so as to facilitate osseo-integration of the surrounding bone into the peg.

And in one preferred form of the invention, collars 195 comprise a porous coating so as to facilitate osseo-integration of the surrounding bone with the collar.

In use, when novel base construct 105 is to be installed in the patient, the top end of the tibia is resected, and a template 202 (FIG. 11A) is positioned against the resected tibia. Template 202 is used to form holes 203 in the resected tibia to receive pegs 160 and, if desired, to form holes in the resected tibia to receive stem 150 and/or post 155. Preferably the axes of holes 203 formed in the resected tibia to receive pegs 160 are offset from the axes of the threaded bores 190 extending through baseplate 115 so that, when pegs 160 are thereafter screwed into threaded bores 190 (FIG. 11B), elongated bodies 165 of pegs 160 create a compressive force against the surrounding bone along the inside edge of the pegs such that compressive forces are generated between the underside of the baseplate and the superior surface of the resected tibia. By way of example but not limitation, if threaded bores 190 are set at an angle A to the plane of baseplate 115 (FIG. 11B), then holes 202 may preferably be set at an angle B relative to the plane of baseplate 115 (FIG. 11A), with B>A, in order to create compression between baseplate 115 and the resected tibia when pegs 160 are screwed into threaded bores 190.

Next, the template is removed and pegs 160 are inserted into their seats formed in the resected tibia. Then baseplate 115 is positioned against the resected tibia so that the exterior threads 180 of pegs 160 engage the threaded bores 190 of baseplate 115. Then a driver is used to screw pegs 160, retrograde, into threaded bores 190 of baseplate 115. It will be appreciated that pegs 160 will then extend at a non-perpendicular angle to the plane of baseplate 115, and stem 150 and post 155 will extend at a perpendicular angle to the plane of baseplate 115, thereby ensuring that at least two of the fixation elements 130 extend downwardly from bottom surface 125 of baseplate 115 in a non-parallel manner. By virtue of the fact that at least two of the fixation elements 130 extend downwardly from bottom surface 125 of baseplate 115 in a non-parallel manner, baseplate 115 will be cementlessly secured to the tibia while effectively eliminating micromotion between the prosthesis and the tibia.

Significantly, by forming the seats 203 for pegs 160 so that their axes are offset from the axes of the threaded bores 190 extending through baseplate 115, i.e., in the manner shown in FIGS. 11A and 11B, where B>A, pegs 160 can create a compressive force against the surrounding bone when pegs 160 are screwed into threaded bores 190 of baseplate 115, whereby to create a compressive force holding baseplate 115 against the resected tibia.

A bearing construct (e.g., the aforementioned polyethylene bearing construct 35) may then be locked into place on baseplate 115 (e.g., such as by using a “tongue-in-groove” locking mechanism or another locking mechanism), whereby to complete installation of novel base construct 105 in the patient.

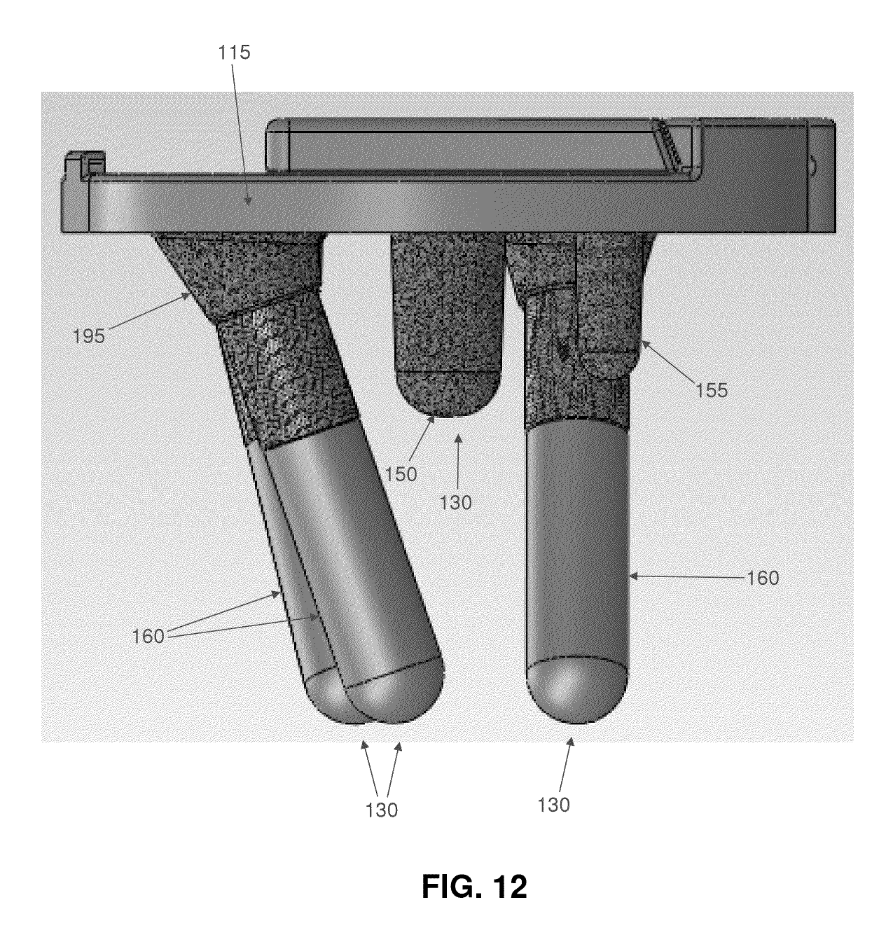

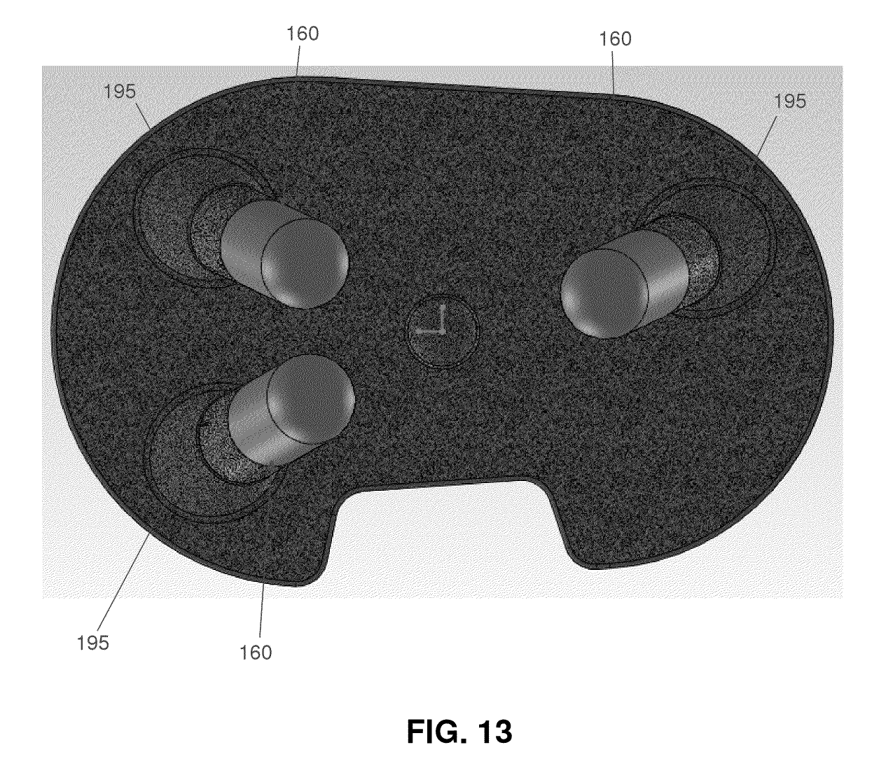

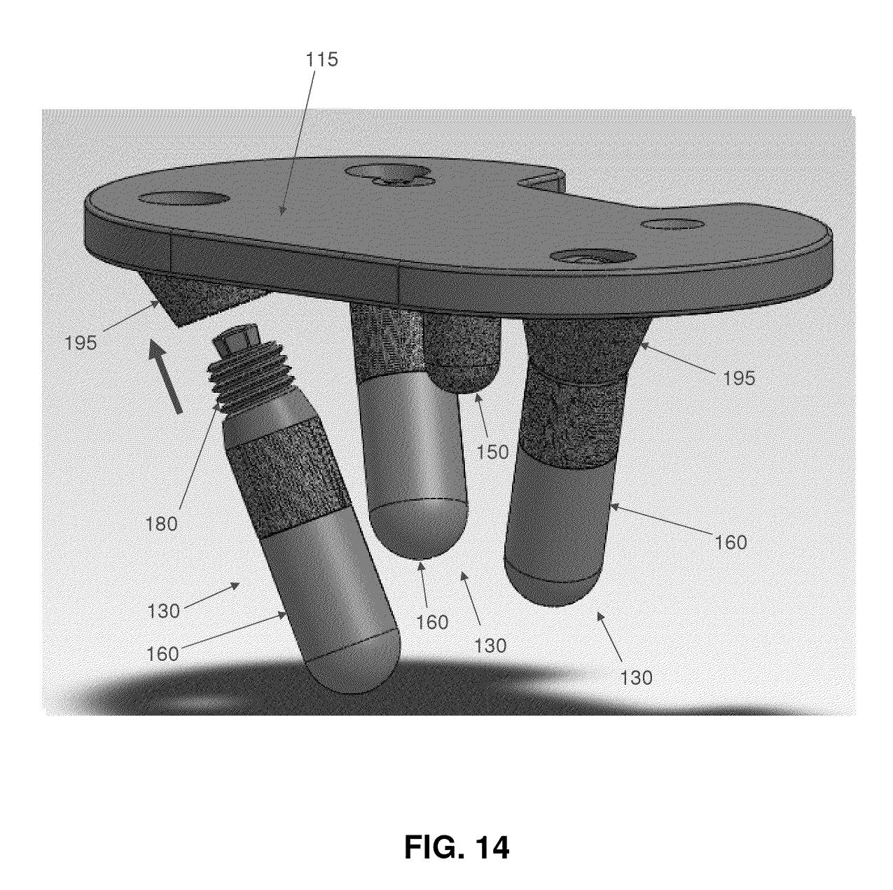

It should be appreciated that, if desired, and looking now at FIGS. 12-14, pegs 160 may have a porous coating formed along only a portion of their lengths, e.g., on a more proximal portion of their elongated bodies. In such a construction, the more distal portions of pegs 160 may have a relatively smooth exterior surface, whereby to facilitate disposition of pegs 160 in the resected tibia.

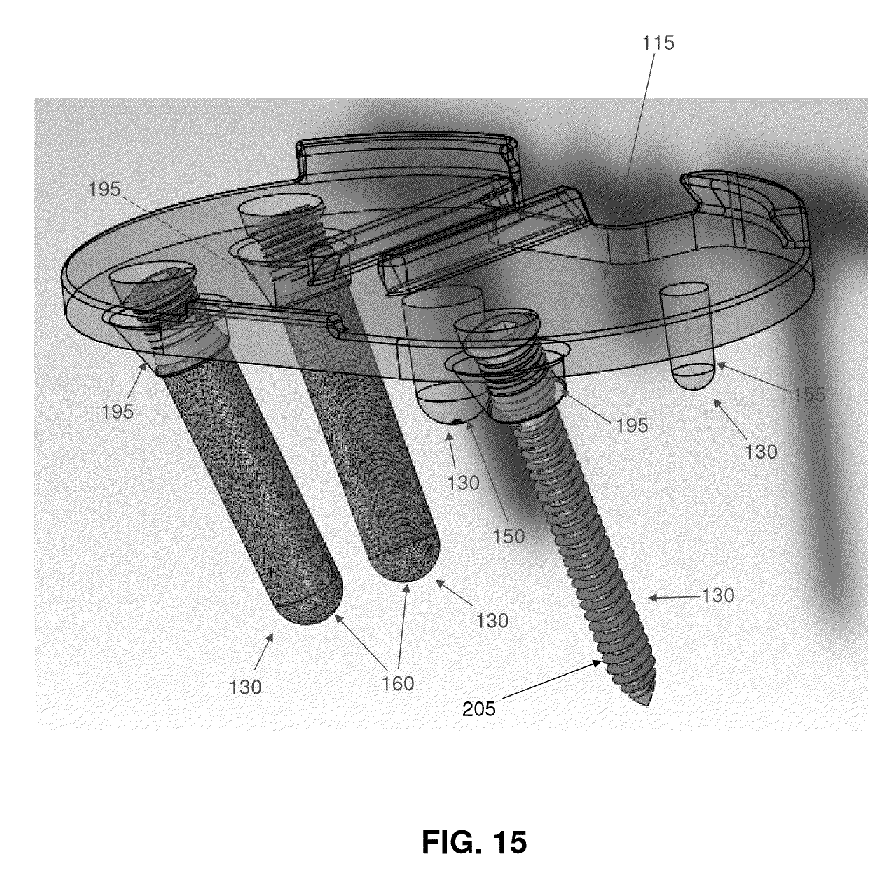

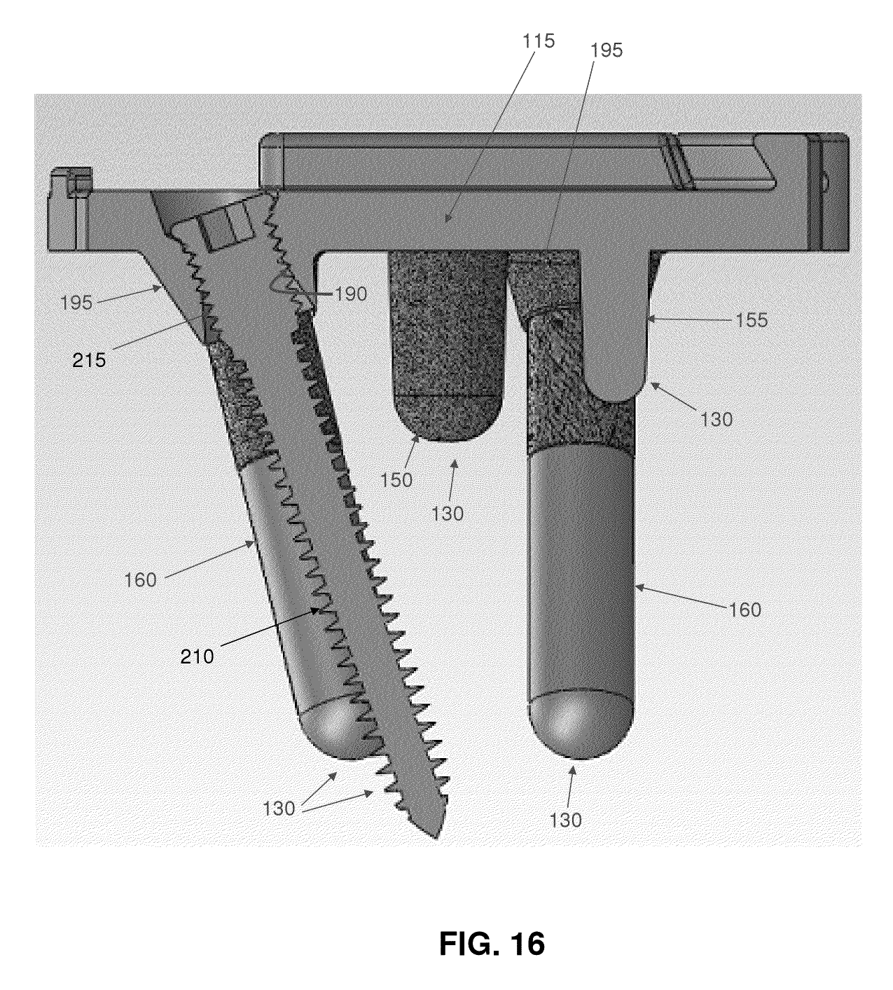

It should also be appreciated that, if desired, and looking now at FIGS. 15 and 16, one or more pegs 160 may be replaced by a bone screw 205. More particularly, in this form of the invention, bone screw 205 preferably comprises distal threads 210 for passing through threaded bore 190 of baseplate 115 and engaging the resected tibia below the baseplate, and proximal threads 215 for engaging threaded bore 190 of baseplate 115, whereby to create downward compression between the baseplate and the bone. The head of the screw is unable to pass through the threaded hole, creating tension along the long axis of the screw as distal threads 210 work to advance the screw through the bone. It will be appreciated that, in this form of the invention, bone screws 205 are set antegrade, i.e., they are first passed through baseplate 115 and then into the resected tibia.

### Modifications

While the present invention has been described in terms of certain exemplary preferred embodiments, it will be readily understood and appreciated by those skilled in the art that it is not so limited, and that many additions, deletions and modifications may be made to the preferred embodiments discussed herein without departing from the scope of the invention.

What is claimed is:

1. Apparatus for reconstructing a joint, the apparatus comprising:
an implant body having a bone contacting surface; and
a plurality of fixation elements secured to the implant body and extending into the host bone at a plurality of angles, wherein all angles are not equal to one another, so as to create immediate stability between the implant body and the host bone.

2. Apparatus according to claim 1 wherein at least one of the fixation elements is removably secured to the implant body.

3. Apparatus according to claim 1 wherein at least one of the fixation elements is fixedly secured to the implant body.

4. Apparatus according to claim 1 wherein at least one of the fixation elements is secured to the implant body in a retrograde manner.

5. Apparatus according to claim 1 wherein at least one of the fixation elements is secured to the implant body in an antegrade manner.

6. Apparatus according to claim 1 wherein the implant body comprises a porous coating.

7. Apparatus according to claim 1 wherein at least one of the fixation elements comprises a porous coating.

8. Apparatus according to claim 1 wherein the fixation elements are selected from the group consisting of a stem, a post, a peg and a screw.

9. A method for reconstructing a joint, the method comprising:
providing apparatus comprising:
an implant body having a bone contacting surface; and
a plurality of fixation elements to be secured to the implant body and extend into the host bone at a plurality of angles, wherein all angles are not equal to one another, so as to create immediate stability between the implant body and the host bone;
positioning the implant body against a host bone; and
creating immediate stability between the implant body and the host bone using the plurality of fixation elements.

10. A method according to claim 9 wherein at least one of the fixation elements is removably secured to the implant body after the implant body is positioned against the host bone.

11. A method according to claim 9 wherein at least one of the fixation elements is fixedly secured to the implant body before the implant body is positioned against the host bone.

12. A method according to claim 9 wherein at least one of the fixation elements is secured to the implant body in a retrograde manner.

13. A method according to claim 12 wherein at least one of the fixation elements is disposed in the host bone before the implant body is positioned against the host bone.

14. A method according to claim 13 wherein the implant body comprises a bore for receiving a fixation element, and further wherein the fixation element is disposed in the host bone with a disposition which is offset from the disposition of the bore, such that when the fixation element is received in the bore, the fixation element applies a compressive force against the host bone, which in turn creates a compressive force across the bone-implant interface.

15. A method according to claim 9 wherein at least one of the fixation elements is secured to the implant body in an antegrade manner after the implant body is positioned against the host bone.

16. A method according to claim 9 wherein the implant body comprises a porous coating.

17. A method according to claim 9 wherein at least one of the fixation elements comprises a porous coating.

18. A method according to claim 9 wherein the fixation elements are selected from the group consisting of a stem, a post, a peg and a screw.

19. Apparatus for reconstructing the knee joint, the apparatus comprising:
a tibial baseplate; and
at least two fixation elements for securing the tibial baseplate to a bone, wherein the at least two fixation elements are secured to the tibial baseplate and extend into the tibia at angles which are not parallel to one another.

20. A method for reconstructing the knee joint, the method comprising:
providing apparatus comprising:
a tibial baseplate; and
at least two fixation elements for securing the tibial baseplate to a bone, wherein the at least two fixation elements are secured to the tibial baseplate and extend into the tibia at angles which are not parallel to one another;
positioning the tibial baseplate against a resected tibia; and
securing the tibial baseplate to the resected tibia using the at least two fixation elements, wherein the at least two fixation elements are secured to the tibial baseplate and extend into the tibia at angles which are not parallel to one another.

21. A method according to claim 20 wherein the at least two fixation elements are inserted into the host bone before the tibial baseplate is positioned against the resected tibia, and further wherein the at least two fixation elements are secured to the tibial baseplate after the tibial baseplate has been positioned against the resected tibia.
